SEQUENCE LISTING (1 sequence)

SEQ ID NO: 1 - protein, 734 aa, synthetic construct
  REGION 1..734: note = AAV2 VP3 common region of the capsid protein
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRN

(19) United States
(12) Patent Application Publication
(10) Pub. No.: US 2023/0338582 A1
(43) Pub. Date: Oct. 26, 2023

(54) Methods of Treating Human X-Linked Retinoschisis Using Gene Therapy

(71) Applicant: TEAMEDON INTERNATIONAL, INC., Rockville, MD (US)

(72) Inventor: Xueyan Peter Mu, Rockville, MD (US)

(21) Appl. No.: 18/161,655

(22) Filed: Jan. 30, 2023

Related U.S. Application Data
(63) Continuation-in-part of application No. PCT/US2022/079195, filed on Nov. 3, 2022.
(60) Provisional application No. 63/275,351, filed on Nov. 3, 2021.

(51) Int. Cl. A61K 48/00; A61K 38/17; A61P 27/02

(52) U.S. Cl. CPC A61K 48/0066; A61K 38/1709; A61K 48/0041; A61P 27/02

(57) ABSTRACT

A method of treating X-linked juvenile retinoschisis (XLRS) in a human subject includes subretinally delivering to the human subject a therapeutically effective amount of an rAAV vector. The rAAV vector includes a nucleic acid sequence comprising coding sequence for human RS1 protein. The rAAV vector can further include a mutated AAV2 VP3 capsid protein having a phenylalanine (F) for tyrosine (Y) substitution at each of the positions corresponding to Y444, Y500 and Y730 in a wild type AAV2 VP3 capsid protein.

### INCORPORATION OF SEQUENCE LISTING

This application includes a Sequence Listing which is being submitted in XML format, named “TEAM002US.xml”, which is 3 KB in size and created on Jan. 30, 2023. The contents of the Sequence Listing are incorporated herein by reference in their entirety.

### BACKGROUND

X-linked juvenile retinoschisis (XLRS) is a recessive degenerative disease of the central retina affecting only males with a worldwide prevalence estimated at 1/5000-1/25,000. XLRS is caused by mutations in the gene that encodes a protein called retinoschisin (RS1), a cell-surface adhesion molecule expressed by photoreceptor and bipolar cells of the retina. The protein has two conserved sequence motifs, an initial signal sequence targets the protein for secretion and the larger discoidin domain is implicated in cell adhesion. RS1 helps to maintain the structural organization of the retinal cell layers and promotes visual signal transduction.

Affected individuals have a relatively normal a-wave in the electroretinogram (ERG), while the b-wave is nearly or totally absent. Another hallmark of XLRS is the localized splitting of the central retina, which develops primarily in the fovea, but can also be present in the peripheral retina. These cystoid cavities may coalesce, leading to further visual acuity loss.

There is no specific treatment for XLRS therefore this represents an important unmet medical need. Anecdotal reports suggest that topical carbonic anhydrase inhibitors may provide some reduction in degree of schisis detected by OCT and improvement in visual acuity in some but not all patients. No products have been approved by regulatory agencies for treatment of this condition. Thus far, treatment of XLRS has been limited to the prescription of low-vision aids. Surgical interventions benefit the patient only in rare cases.

Recombinant AAV (rAAV) vectors have been developed by deleting the viral rep and cap genes, inserting a transgene expression cassette between the ITRs, and packaging the vector DNA into AAV capsids in a packaging cell. rAAV vectors are uniquely suitable for in vivo gene therapy because they are non-toxic, highly efficient at transducing a wide variety of non-dividing cell types, and persist for long periods, primarily in episomal form, resulting in long-term expression of the transgene. rAAV vectors have been effective for treatment of a wide variety of animal models of genetic diseases, including retinal diseases, hemophilia, muscular dystrophy, lysosomal storage disorders and diseases of the central nervous system.

Mice deficient in retinoschisin have been developed and used to obtain insight into the role of retinoschisin in retinal structure, function, and pathology. Studies in these murine models of XLRS have shown that recombinant adeno-associated virus (rAAV) gene therapy vectors expressing normal RS1 can provide significant restoration of retinal structure and function in RS1-deficient mice.

Previous clinical trials using gene therapy by AAV-RS1 gene vector to treat XLRS patients using intravitreal injection route were not successful. This lack of efficacy may be due to the dilution of the administered vector in the vitreous humor. There remains a need to find an effective therapy to treat XLRS patients.

### SUMMARY

The present disclosure provides a method of treating X-linked juvenile retinoschisis (XLRS) in a human subject by subretinally delivering to the human subject a therapeutically effective amount of an rAAV vector, the rAAV vector comprising a nucleic acid sequence comprising a coding sequence for human RS1 protein. The rAAV vector can further include a mutated AAV2 VP3 capsid protein comprising phenylalanine (F) for tyrosine (Y) substitutions at each of the positions corresponding to Y444, Y500 and Y730 in a wild type AAV2 VP3 capsid protein. The nucleic acid sequence of the rAAV vector can also further include a CMV enhancer sequence, and/or chicken beta actin promoter sequence. In specific embodiments, the rAAV vector is rAAV2tYF-CB-hRS1 as described herein.

### BRIEF DESCRIPTION OF THE DRAWINGS

FIG. 1 shows the organization of the elements in the DNA of the rAAV2tYF-CB-hRS1 vector.

FIG. 2 shows epifluorescence IHC image of an animal that has been intravitreally injected with AAV2tYF-CB-GFP.

FIG. 3 shows epifluorescence image of an animal that has been subretinally injected with AAV2tYF-CB-GFP.

FIG. 4 is a light-adapted 5 Hz flicker waveform for a mouse treated with rAAV2tYF-CB-hRS1 at mid dose 3E8 vg/μL in one eye (left panel) vs its untreated fellow eye (right panel), which show rAAV2tYF-CB-hRS1 markedly improved cone function.

FIG. 5 is a light-adapted 3.0 ERG waveform for a mouse treated with rAAV2tYF-CB-hRS1 at mid dose 3E8 vg/μL in one eye (left panel) vs its untreated fellow eye (right panel).

FIG. 6 is a light-adapted 3.0 oscillatory potentials waveform for a mouse treated with rAAV2tYF-CB-hRS1 at mid dose 3E8 vg/μL in one eye (left panel) vs its untreated fellow eye (right panel).

FIG. 7 is a dark-adapted 3.0 ERG waveform (standard combined response, rods and cones) for a mouse treated with rAAV2tYF-CB-hRS1 at mid dose 3E8 vg/μL in one eye (left panel) vs its untreated fellow eye (right panel).

FIG. 8 is a dark-adapted 3.0 oscillatory potentials waveform for a mouse treated with rAAV2tYF-CB-hRS1 at mid dose 3E8 vg/μL in one eye (left panel) vs its untreated fellow eye (right panel).

FIG. 9 is a dark-adapted 0.01 ERG waveform for a mouse treated with rAAV2tYF-CB-hRS1 at mid dose 3E8 vg/μL in one eye (left panel) vs its untreated fellow eye (right panel).

FIGS. 10A-10F show the effect of rAAV2tYF-CB-hRS1 on electroretinogram (ERG) after one month of dosing via subretinal injection. FIG. 10A: b-wave of dark-adapted 0.01 ERG; FIG. 10B: a-wave of dark-adapted 3.0 ERG waveform (standard combined response); FIG. 10C: b-wave of dark-adapted 3.0 ERG waveform (standard combined response); FIG. 10D: a-wave of light-adapted 3.0 ERG waveform; FIG. 10E: b-wave of light-adapted 3.0 ERG waveform; FIG. 10F: P1-wave of light-adapted 5 Hz flicker waveform. (n=12 for untreated group, n=3 for all other groups).

### DETAILED DESCRIPTION

Illustrative embodiments of the invention are described below. In the interest of clarity, not all features of an actual implementation are described in this specification. It should be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

In accordance with the present invention various genetic-modifying entities may be used individually or in combination to achieve the desired results. These entities may include naked natural or modified nucleic acid or peptides. Such modifications may contain carbohydrates including fatty acids and/or sugars. As used herein, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, nucleotides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of clarity related to the present invention, terms are defined below:
A, an: In accordance with long standing patent law convention, the words “a” and “an” when used in this application, including the claims, denotes “one or more.”
Expression: The combination of intracellular processes, including transcription and translation undergone by a polynucleotide such as a structural gene to synthesize the encoded peptide or polypeptide.
Promoter: a term used to generally describe the region or regions of a nucleic acid sequence that regulates transcription.
Regulatory Element: a term used to generally describe the region or regions of a nucleic acid sequence that regulates transcription. Exemplary regulatory elements include, but are not limited to, enhancers, post-transcriptional elements, transcriptional control sequences, and such like.
Structural gene: A polynucleotide, such as a gene, that is expressed to produce an encoded peptide, polypeptide, protein, ribozyme, catalytic RNA molecule, siRNA, or antisense molecule.
Transformation: A process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.
Transformed cell: A host cell whose nucleic acid complement has been altered by the introduction of one or more exogenous polynucleotides into that cell.
Transgenic cell: Any cell derived or regenerated from a transformed cell or derived from a transgenic cell, or from the progeny or offspring of any generation of such a transformed host cell.
Vector: A nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human, and in particular, when administered to the human eye. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

As used herein, the term “operatively linked” means that a promoter is connected to a functional RNA in such a way that the transcription of that functional RNA is controlled and regulated by that promoter. Means for operatively linking a promoter to a functional RNA are well known in the art.

### Adeno-Associated Viral Vectors:

The present invention provides methods of administering viral vectors comprising nucleic acids encoding human RS1 protein.

Adeno-associated virus (AAV) is a small (25-nm), nonenveloped virus that packages a linear single-stranded DNA genome of 4.7 Kb. The small size of the AAV genome and concerns about potential effects of Rep on the expression of cellular genes led to the construction of AAV vectors that do not encode Rep and that lack the cis-active IEE, which is required for frequent site-specific integration. The ITRs are kept because they are the cis signals required for packaging. Thus, current recombinant AAV (rAAV) vectors persist primarily as extrachromosomal elements.

A variety of recombinant adeno-associated viral vectors (rAAV) may be used to deliver genes of interest to a cell and to effect the expression of a gene of interest, e.g., a gene encoding hRS1 in a target cell. At times herein, “transgene” is used to refer to a polynucleotide encoding a polypeptide of interest, wherein the polynucleotide is encapsidated in a viral vector (e.g., rAAV).

Adeno-associated viruses are small, single-stranded DNA viruses, which require helper virus to facilitate efficient replication. The 4.7-kb genome of AAV is characterized by two inverted terminal repeats (ITR) and two open reading frames, which encode the Rep proteins and Cap proteins, respectively. The Rep reading frame encodes four proteins of molecular weight 78 kDa, 68 kDa, 52 kDa, and 40 kDa. These proteins function mainly in regulating AAV replication, and rescue and integration of the AAV into a host cell's chromosomes. The Cap reading frame encodes three structural proteins of molecular weight 85 kDa (VP1), 72 kDa (VP2), and 61 kDa (VP3) (Berns), which form the virion capsid. More than 80% of total proteins in AAV virion comprise VP3.

The genome of rAAV is generally comprised of: (1) a 5′ adeno-associated virus ITR, (2) a coding sequence (e.g., transgene) for the desired gene product (e.g., hRS1 protein) operatively linked to a sequence that regulates its expression in a cell (e.g., a promoter sequence such as a mGluR6 or fragment thereof), and (3) a 3′ adeno-associated virus inverted terminal repeat. In addition, the rAAV vector may preferably contain a polyadenylation sequence.

Generally, rAAV vectors have one copy of the AAV ITR at each end of the transgene or gene of interest, in order to allow replication, packaging, and efficient integration into cell chromosomes. The ITR consists of nucleotides 1 to 145 at the 5′-end of the AAV DNA genome, and nucleotides 4681 to 4536 (i.e., the same sequence) at the 3′-end of the AAV DNA genome. The rAAV vector may also include at least 10 nucleotides following the end of the ITR (i.e., a portion of the “D region”).

The transgene sequence (e.g., the polynucleotide encoding hRS1) can be of about 2- to 5-kb in length or longer or shorter lengths of bases (or alternatively, the transgene may additionally contain a “stuffer” or “filler” sequence to bring the total size of the nucleic acid sequence between the two ITRs to between 2 and 5 kb. Alternatively, the transgene may be composed of repeated copies of the same or similar heterologous sequence several times, or several different heterologous sequences.

Recombinant AAV vectors of the present invention may be generated from a variety of adeno-associated viruses, including for example, any of serotypes 1 through 12, as described herein. For example, ITRs from any AAV serotype are expected to have similar structures and functions with regard to replication, integration, excision and transcriptional mechanisms.

In some embodiments, a cell-type specific promoter (or other regulatory sequence such as an enhancer) is employed to drive expression of a gene of interest. Representative examples of suitable promoters in this regard include a CBA promoter (chicken β-actin), CMV promoter, RSV promoter, SV40 promoter, MoMLV promoter, or derivatives, mutants and/or fragments thereof. Promoters and other regulatory sequences are further described herein.

Other promoters that may similarly be utilized within the context of the present invention include cell or tissue specific promoters (e.g., a rod, cone, or ganglia derived promoter), or inducible promoters. Representative examples of suitable inducible promoters include inducible promoters sensitive to an antibiotic, e.g., tetracycline-responsive promoters such as “tet-on” and/or “tet-off” promoters. Inducible promoters may also include promoters sensitive to chemicals other than antibiotics.

The rAAV vector may also contain additional sequences, for example from an adenovirus, which assist in effecting a desired function for the vector. Such sequences include, for example, those that assist in packaging the rAAV vector into virus particles.

Packaging cell lines suitable for producing adeno-associated viral vectors may be accomplished given available techniques (see e.g., U.S. Pat. No. 5,872,005). Methods for constructing and packaging rAA7I vectors are described in, for example, PCT Intl. Pat. Appl. Publ. No. WO 00/54813.

Flanking the rep and cap open reading frames at the 5′ and 3′ ends are 145-bp inverted terminal repeats (ITRs), the first 125 bp of which are capable of forming Y- or T-shaped duplex structures. The two ITRs are the only cis elements essential for AAV replication, rescue, packaging and integration of the AAV genome. There are two conformations of AAV ITRs called “flip” and “flop.” These differences in conformation originated from the replication model of adeno-associated virus, which uses the ITR to initiate and reinitiate the replication (R. O. Snyder et al., J. Viral., 67:6096-6104; 1993; K. I. Berns, Microbiol. Rev., 54:316-329; 1990). The entire rep and cap domains can be excised and replaced with a therapeutic or reporter transgene.

In some embodiments, self-complementary AAV vectors are used. Self-complementary vectors have been developed to circumvent rate-limiting second-strand synthesis in single-stranded AAV vector genomes and to facilitate robust transgene expression at a minimal dose. In specific embodiments, a self-complementary AAV of any serotype or hybrid serotype or mutant serotype, or mutant hybrid serotype increases expression of hRS1 and variants thereof by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, or more than 200%, when compared to a non-self-complementary rAAV of the same serotype.

In one aspect, the present disclosure provides methods of using an Adeno-associated Virus Vector Expressing Retinoschisin in treating Patients with X-linked Retinoschisis, or AAV-RS1. In some embodiments, the AAV is of a serotype selected from the group consisting of AAV1, AAV2, AAV3, AAV4, AAV5, AAV6, AAV7, AAV8, AAV9, AAV10, AAV11, AAV12, and hybrids thereof. In other specific embodiments, the AAV is recombinant AAV of a combinatorial hybrid of 2, 3, 4, 5, 6, 7, 8, 9, 10, 11, 12, 13, 14, 15 or more serotypes or mutants thereof. In some embodiments, the AAV vector is encapsulated in an AAV capsid protein which comprises at least one mutated tyrosine residue. The mutated tyrosine residue can be selected from the group consisting of Y252F, Y272F, Y444F, Y500F, Y700F, Y704F, Y730F, Y275F, Y281F, Y508F, Y576F, Y612G, Y673F, and Y720F. In a specific embodiment, the mutated capsid protein comprises one or more tyrosine residues, each mutated to a phenylalanine residue.

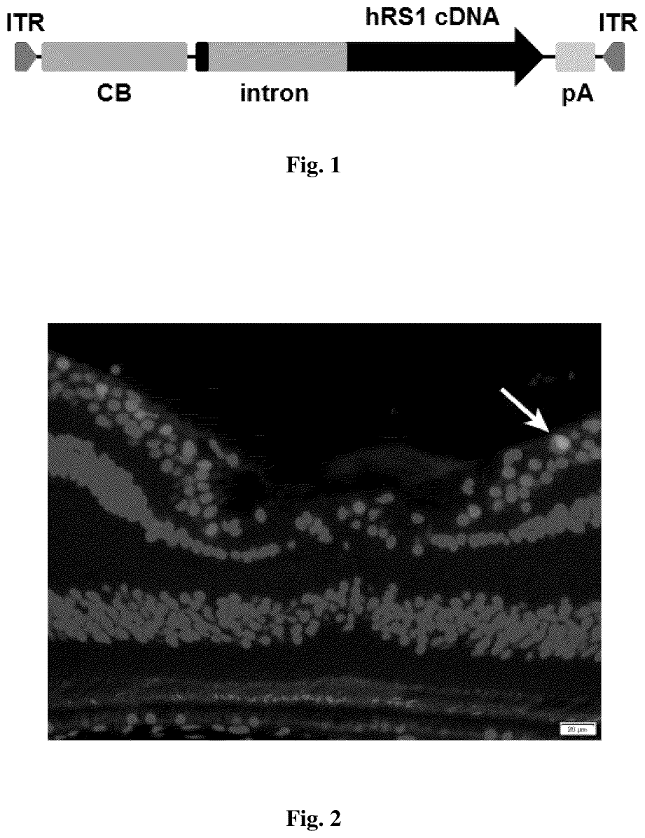

As an example, rAAV2tYF-CB-hRS1 is a replication-incompetent, recombinant adeno-associated virus (rAAV) vector that expresses the retinoschisin (RS1) protein after the vector enters retinal cells. rAAV2tYF-CB-hRS1 cDNA encodes the human retinoschisin (RS1) protein. The vector contains AAV serotype 2 inverted terminal repeats and an expression cassette consisting of a CMV enhancer, chicken beta actin promoter, the human RS1 cDNA and an SV40 polyadenylation sequence. The organization of the elements in the DNA of the rAAV2tYF-CB-hRS1 vector is depicted in FIG. 1, where ITR denotes inverted terminal repeat, CB denotes CMV enhancer and chicken beta actin promoter, RS1 denotes retinoschisin, pA denotes a polyadenylation sequence. The vector DNA is packaged in an AAV2 capsid referred to as AAV2tYF which contains tyrosine to phenylalanine (YF) mutations in three tyrosine residues at each of the positions corresponding to Y444, Y500 and Y730 (VP1 numbering) in a wild type AAV2 VP3 common region of the capsid protein. The sequence of the wild type AAV2 VP1 capsid protein is provided below:

(SEQ ID NO: 1)
MAADGYLPDWLEDTLSEGIRQWWKLKPGPPPPKPAERHKDD
SRGLVLPGYKYLGPFNGLDKGEPVNEADAAALEHDKAYDRQ
LDSGDNPYLKYNHADAEFQERLKEDTSFGGNLGRAVFQAKK
RVLEPLGLVEEPVKTAPGKKRPVEHSPVEPDSSSGTGKAGQQ
PARKRLNFGQTGDADSVPDPQPLGQPPAAPSGLGTNTMATGS
GAPMADNNEGADGVGNSSGNWHCDSTWMGDRVITTSTRTW
ALPTYNNHLYKQISSQSGASNDNHYFGYSTPWGYFDFNRFHC
HFSPRDWQRLINNNWGFRPKRLNFKLFNIQVKEVTQNDGTTT
IANNLTSTVQVFTDSEYQLPYVLGSAHQGCLPPFPADVFMVP
QYGYLTLNNGSQAVGRSSFYCLEYFPSQMLRTGNNFTFSYTF
EDVPFHSSYAHSQSLDRLMNPLIDQYLYYLSRTNTPSGTTTQS
RLQFSQAGASDIRDQSRNWLPGPCYRQQRVSKTSADNNNSE
YSWTGATKYHLNGRDSLVNPGPAMASHKDDEEKFFPQSGVL
IFGKQGSEKTNVDIEKVMITDEEEIRTTNPVATEQYGSVSTNL
QRGNRQAATADVNTQGVLPGMVWQDRDVYLQGPIWAKIPH
TDGHFHPSPLMGGFGLKHPPPQILIKNTPVPANPSTTFSAAKF
ASFITQYSTGQVSVEIEWELQKENSKRWNPEIQYTSNYNKSV
NVDFTVDTNGVYSEPRPIGTRYLTRN

The rAAV2tYF-CB-hRS1 vector can be produced using a recombinant herpes simplex virus complementation system in suspension-cultured baby hamster kidney cells. See Ye G J, Budzynski E, Sonnentag P, et al. Safety and biodistribution evaluation in cynomolgus macaques of rAAV2tYF-CB-hRS1, a recombinant adeno-associated virus vector expressing retinoschisin. Hum Gene Ther Clin Dev 2015; 3:165-176. Details are provided in the Thomas D L, Wang L, Niamke J, et al. Scalable recombinant adeno-associated virus production using recombinant herpes simplex virus type 1 coinfection of suspension-adapted mammalian cells. Hum Gene Ther 2009; 20:861-870. The disclosures of these references are incorporated by reference in their entireties.

For example, two rHSV helper viruses, one containing the AAV2 rep and AAV2tYF cap genes and the other containing the hRS1 expression cassette, can be used to coinfect sBHK cells grown in serum-free medium. The cells can then be lysed with Triton X-100 detergent and treated with Benzonase. Cell lysate containing the AAV vector can be clarified by filtration and purified by AVB Sepharose (GE Life Sciences) affinity chromatography followed by CIM SO3⁻ (BIA Separations) cation-exchange chromatography, and eluted in concentrated balanced salt solution containing 0.014% (v/v) Tween-20 (BSST). The purified bulk can then be concentrated and buffered exchanged to 1×BSST (drug substance) and sterile (0.2 μm) filtered to generate drug product. The vector can be further concentrated, as needed, using a 100 kDa MWCO Ultra centrifugal filter unit (EMD Millipore), and re-filtered (0.2 μm) to generate drug product sublots of specific concentrations. It can be stored frozen and thawed and diluted to the appropriate concentration immediately before administration.

### Pharmaceutical Compositions

Gene delivery vectors can be prepared as a pharmaceutically acceptable composition suitable for administration. In general, such pharmaceutical compositions comprise an amount of a gene delivery vector suitable for delivery of protein-encoding polynucleotide to a cell of the eye for expression of a therapeutically effective amount of the hRS1 protein, combined with a pharmaceutically acceptable carrier or excipient. Preferably, the pharmaceutically acceptable carrier is suitable for intraocular administration. Exemplary pharmaceutically acceptable carriers include, but are not necessarily limited to, saline or a buffered saline solution (e.g., phosphate-buffered saline).

Various pharmaceutically acceptable excipients are well known in the art. As used herein, “pharmaceutically-acceptable excipient” includes any material, which, when combined with an active ingredient of a composition, allows the ingredient to retain biological activity, preferably without causing disruptive reactions with the subject's immune system or adversely affecting the tissues surrounding the site of administration (e.g., within the eye).

Exemplary pharmaceutically carriers include sterile aqueous of non-aqueous solutions, suspensions, and emulsions. Examples include, but are not limited to, any of the standard pharmaceutical excipients such as a saline, buffered saline (e.g., phosphate buffered saline), water, emulsions such as oil/water emulsion, and various types of wetting agents.

Examples of non-aqueous solvents are propylene glycol, polyethylene glycol, hyaluronic acid, vegetable oils such as olive oil, and injectable organic esters such as ethyl oleate.

Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles can include fluid and nutrient replenishers, electrolyte replenishers (such as those based on Ringer's dextrose), and the like.

A composition of gene delivery vector of the invention may also be lyophilized using means well known in the art, for subsequent reconstitution and use according to the invention. Where the vector is to be delivered without being encapsulated in a viral particle (e.g., as “naked” polynucleotide), formulations for liposomal delivery, and formulations comprising microencapsulated polynucleotides, may also be of interest.

Compositions comprising excipients are formulated by well-known conventional methods (see, for example, Remington's Pharmaceutical Sciences, Chapter 43, 14th Ed., Mack Publishing Co., Easton, Pa., USA).

In general, the pharmaceutical compositions can be prepared in various forms, preferably a form compatible with intraocular administration. Stabilizing agents, wetting and emulsifying agents, salts for varying the osmotic pressure or buffers for securing an adequate pH value may also optionally be present in the pharmaceutical composition.

The amount of gene delivery vector in the pharmaceutical formulations can vary widely, i.e., from less than about 0.1%, usually at or at least about 2% to as much as 20% to 50% or more by weight, and may be selected primarily by fluid volumes, viscosities, etc., in accordance with the particular mode of administration selected.

### Subretinal Administration

A human subject suffering from XLRS can be treated by the compositions of the present application, e.g., rAAV2tYF-CB-hRS1, by introducing the composition into an affected eye of the subject by subretinal administration. The doses of rAAV2tYF-CB-hRS1 can be delivered using a subretinal injection cannula, e.g., with a polyamide micro tip with an inner diameter of 41 gauge. The site of the injection can be identified preoperatively to avoid schisis. On the day of surgery, the injection and resulting bleb can be monitored with intraoperative optical coherence tomography. The site of injection should avoid direct contact with the retinal vasculature or with areas of pathologic features. The entry point along the superior arcade and quality of subretinal injection can be identified under surgical microscope. An example of subretinal injection device is the MedOne MicroDose Injection Device (a 1 mL syringe with adapter and a PolyTip Cannula 25/38 g).

Further, a kit including the compositions of the present application, e.g., rAAV2tYF-CB-hRS1, as well as a delivery device (such as a syringe or cannula described herein), with instructions for use of the kit for subretinal administration to patients provided in physical manual and/or digital files stored on computer readable medium (e.g., CD-ROM, flash drives, etc.). The composition can be pre-loaded in the delivery device, or stored in a separate container from the delivery device.

### Example 1

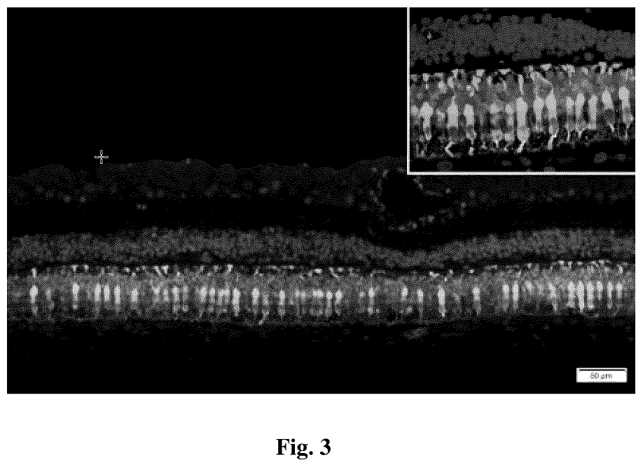

A transduction efficiency study was conducted in normal nonhuman primates using a same vector as in rAAV2tYF-CB-hRS1 but containing a green fluorescent marker protein (GFP) coding sequence (rAAV2tYF-CB-GFP) via intravitreal or subretinal injection. Ten female cynomolgus monkeys were assigned to two groups (4 or 6 females/group), and a dose of 1×10¹¹ μg/eye were administered via intravitreal injection or subretinal injection. Animals were dosed once on Study Day 1. After dosing, animals were observed post-dose for approximately 12 weeks to assess GFP expression. The tolerability and transduction efficiency were also assessed. The study results shown that green fluorescent protein (GFP) fluorescence in intravitreal eyes was mainly limited to some retinal ganglion cells (RGCs) surrounding the fovea and their axons (FIG. 2: Epifluorescence IHC image of animal that had been intravitreally injected with AAV2tYF-CB-GFP. The red/green cone outer segments are labeled red. A single ganglion cell (arrow) shows green staining in its cytoplasm for GFP. Bar=20 μm).

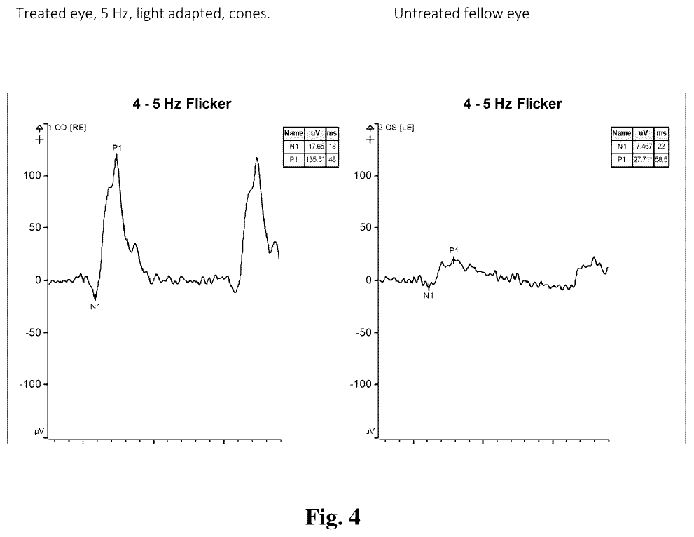

Subretinal administration resulted in marked GFP fluorescence in photoreceptor cells (rods and cones) located over the subretinal delivery site (FIG. 3: Epifluorescence image of animal in the superior periphery at the location of the subretinal bleb. The inset is of the same area at higher magnification taken with a confocal microscope. It is apparent that the cones are more strongly labeled than the rods. The cones are wider than the rods and their nuclei are located near the external limiting membrane. Also, many of their outer segments are labeled red—the reaction product from the anti-red/green cone IHC. Bar=50 μm). Administration of test article via both delivery routes was found to associate with ocular inflammation, which was more severe in intravitreal eyes. The results from this study suggest that subretinal injection of more efficient in transducing photoreceptor cells and causes less ocular inflammation comparing to intravitreal injection.

### Example 2: Safety and Efficacy Study of rAAV2tYF-CB-hRS1 in RS1-KO Mice

The goal of this study was to determine the safety and efficacy of rAAV2tYF-CB-hRS1 in RS1-KO mice via subretinal injection. Twelve (12) RS1-KO mice were divided into 4 groups with 3 animals per group, anesthetized, and 2 microliters of 3E9 vg/μL, 3E8 vg/μL and 3E7 vg/μL rAAV2tYF-CB-hRS1, or diluent were injected subretinally in the right eyes of the animals in each group. The left eyes of the mice served as uninjected controls. At one month after treatment, electroretinogram were performed to evaluate efficacy and safety.

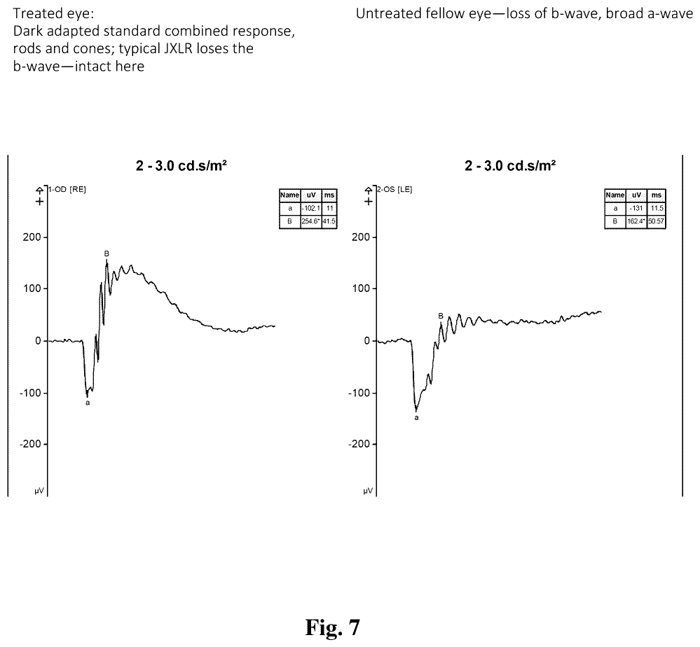

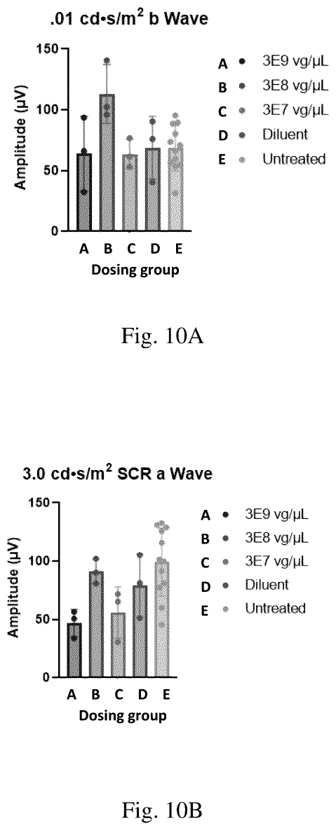

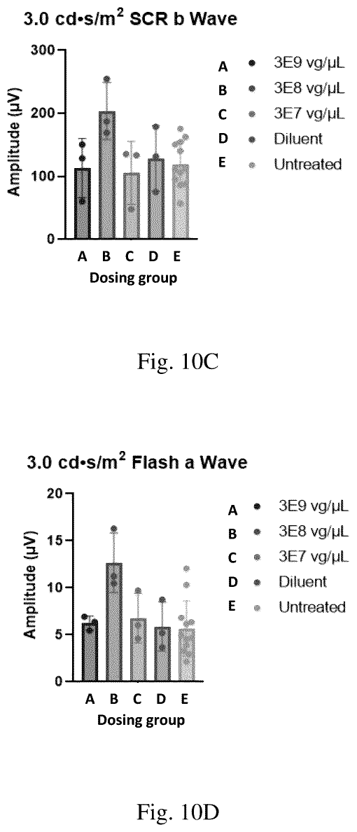

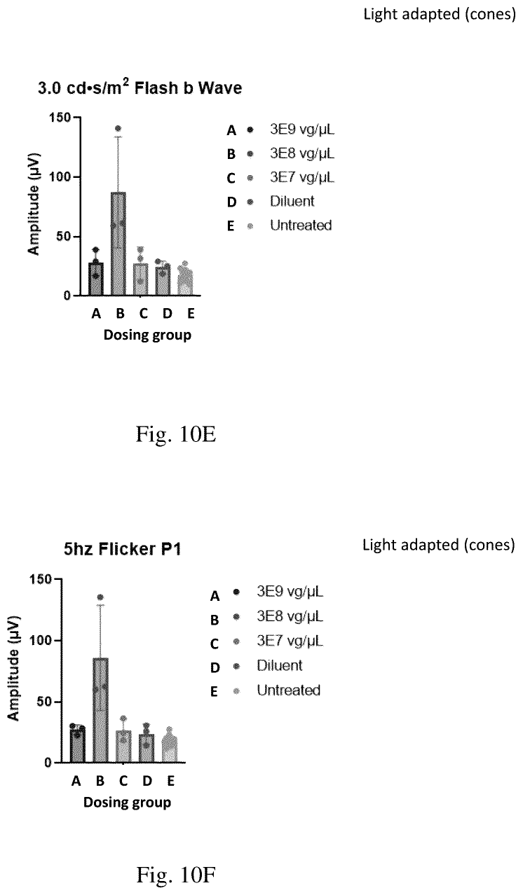

The results show that subretinally delivered rAAV2tYF-CB-hRS1 markedly improved cone function (light adapted ERG) at one month after injection (FIGS. 4, 5 and 6), and there was improvement for rod function and b-wave as well (FIGS. 7, 8, and 9). Mid dose 3E8 vg/μL was shown to be superior to the higher and lower doses (FIGS. 10A-10F).

FIG. 4 is a light-adapted 5 Hz flicker waveform for a mouse treated with rAAV2tYF-CB-hRS1 at mid dose 3E8 vg/μL in one eye (left panel) vs its untreated fellow eye (right panel), which show rAAV2tYF-CB-hRS1 markedly improved cone function.

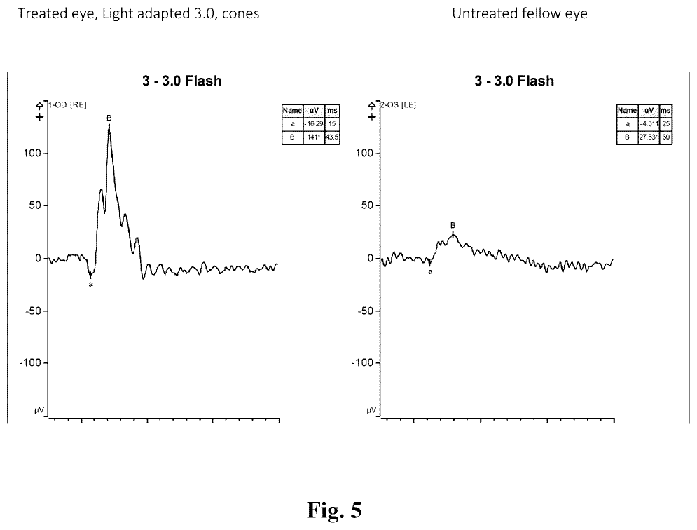

FIG. 5 is a light-adapted 3.0 ERG waveform for a mouse treated with rAAV2tYF-CB-hRS1 at mid dose 3E8 vg/μL in one eye (left panel) vs its untreated fellow eye (right panel), which show rAAV2tYF-CB-hRS1 markedly improved cone function.

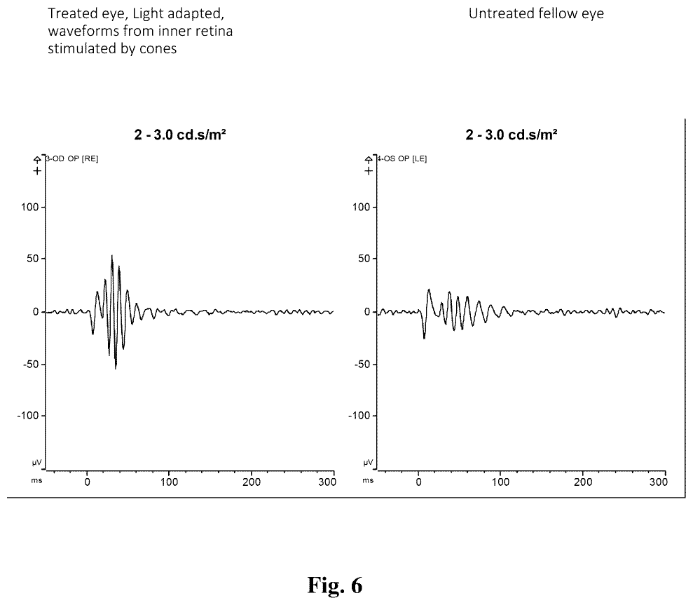

FIG. 6 is a light-adapted 3.0 oscillatory potentials waveform for a mouse treated with rAAV2tYF-CB-hRS1 at mid dose 3E8 vg/μL in one eye (left panel) vs its untreated fellow eye (right panel), which show rAAV2tYF-CB-hRS1 markedly improved cone function.

FIG. 7 is a dark-adapted 3.0 ERG waveform (standard combined response, rods and cones) for a mouse treated with rAAV2tYF-CB-hRS1 at mid dose 3E8 vg/μL in one eye (left panel) vs its untreated fellow eye (right panel), which show rAAV2tYF-CB-hRS1 markedly improved rod and cone function, and b-wave.

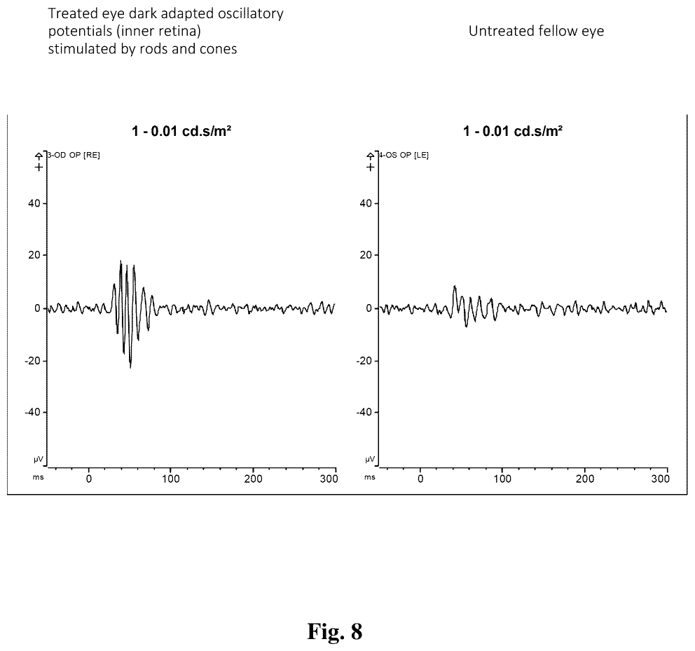

FIG. 8 is a dark-adapted 3.0 oscillatory potentials waveform for a mouse treated with rAAV2tYF-CB-hRS1 at mid dose 3E8 vg/μL in one eye (left panel) vs its untreated fellow eye (right panel), which show rAAV2tYF-CB-hRS1 markedly improved rod and cone function.

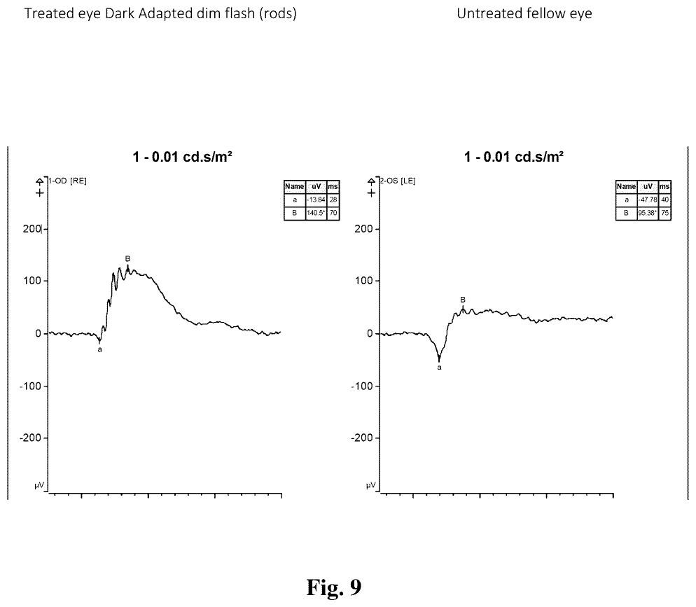

FIG. 9 is a dark-adapted 0.01 ERG waveform for a mouse treated with rAAV2tYF-CB-hRS1 at mid dose 3E8 vg/μL in one eye (left panel) vs its untreated fellow eye (right panel), which show rAAV2tYF-CB-hRS1 markedly improved rod function.

FIGS. 10A-10F show the effect of rAAV2tYF-CB-hRS1 on electroretinogram (ERG) after one month of dosing via subretinal injection. FIG. 10A: b-wave of dark-adapted 0.01 ERG; FIG. 10B: a-wave of dark-adapted 3.0 ERG waveform (standard combined response); FIG. 10C: b-wave of dark-adapted 3.0 ERG waveform (standard combined response); FIG. 10D: a-wave of light-adapted 3.0 ERG waveform; FIG. 10E: b-wave of light-adapted 3.0 ERG waveform; FIG. 10F: P1-wave of light-adapted 5 Hz flicker waveform. (n=12 for untreated group, n=3 for all other groups).

The present invention is not to be limited in scope by the specific embodiments described herein. It will be appreciated that the invention is susceptible to modification, variation and change without departing from the spirit thereof.

1. A method of treating X-linked juvenile retinoschisis (XLRS) in a human subject, comprising:
subretinally delivering to the human subject a therapeutically effective amount of an rAAV vector, the rAAV vector comprising a nucleic acid sequence comprising coding sequence for human RS1 protein.

2. The method of claim 1, wherein the rAAV vector further comprises a mutated AAV2 VP3 capsid protein comprising phenylalanine (F) for tyrosine (Y) substitutions at each of the positions corresponding to Y444, Y500 and Y730 in a wild type AAV2 VP3 capsid protein.

3. The method of claim 1, wherein the nucleic acid sequence of the rAAV vector further comprises a chicken beta actin promoter sequence.

4. The method of claim 1, wherein the nucleic acid sequence of the rAAV vector further comprises a CMV enhancer.

5. The method of any of the foregoing claims, wherein the rAAV vector is rAAV2tYF-CB-hRS1.

6. A method of treating X-linked juvenile retinoschisis (XLRS) in a human subject, comprising:
subretinally delivering to the human subject a pharmaceutical composition comprising rAAV2tYF-CB-hRS1 and a pharmaceutically-acceptable carrier.

7. The method of claim 2, wherein the nucleic acid sequence of the rAAV vector further comprises a chicken beta actin promoter sequence.

8. The method of claim 2, wherein the nucleic acid sequence of the rAAV vector further comprises a CMV enhancer.
